(19) United States
(12) Patent Application Publication
(10) Pub. No.: US 2020/0392581 A1
(43) Pub. Date: Dec. 17, 2020

(54) CHEMOENDOCRINE SCORE (CES) BASED ON PAM50 FOR BREAST CANCER WITH POSITIVE HORMONE RECEPTORS WITH AN INTERMEDIATE RISK OF RECURRENCE

(71) Applicant: GEICAM (Grupo Español de Investigación en Cancer de Mama), San Sebastián de los Reyes (ES)

(72) Inventors: Aleix Prat, Barcelona (ES); Charles M. Perou, Chapel Hill, NC (US); Emilio Alba Conejo, Málaga (ES)

(21) Appl. No.: 16/464,194

(22) PCT Filed: Nov. 27, 2017
(86) PCT No.: PCT/ES2017/000148

§ 371 Date: Aug. 7, 2020

(30) Foreign Application Priority Data
Nov. 28, 2016 (ES) P201601012

(51) Int. Cl. C12Q 1/6886; G16B 20/00; G16B 40/20

(52) U.S. Cl. CPC C12Q 1/6886; C12Q 2600/106; G16B 40/20; G16B 20/00

(57) ABSTRACT

This new method relates to the development of a chemoendocrine score (CES), based on the known analysis PAM50 for predicting whether a patient with breast cancer will respond to chemotherapy or to endocrine therapy, specifically in a patient with HR+/HER2− breast cancer beyond the risk of recurrence (ROR) and PAM50 intrinsic subtypes. Specifically, the clinical utility of this CES predictor is based on the intermediate PAM50 ROR group, the proportion of each CES group (sensitive to endocrine therapy, intermediate, and sensitive to chemotherapy) being greater than 25%.

The new method relates to the field of treatment efficacy prediction in patients with breast cancer. Specifically, this new method relates to chemotherapy or endocrine therapy efficacy prediction in patients with hormone receptor-positive (HR+) and HER2 negative (HR+/HER2−) breast cancer, regardless of the intrinsic subtype and the risk of relapse (ROR), preferably in the case in which the patient is classified as a patient with an intermediate risk of relapse.

### STATE OF THE ART

About 70% of invasive breast cancers are HR+/HER2− at the moment of diagnosis (TCGA: Comprehensive molecular portraits of human breast tumours. Nature 2012; 490:61-70; Prat A, et al. Journal of Clinical Oncology. 2013, 31:203-209). However, HR+/HER2-disease is clinically and biologically heterogeneous and other subclassifications are require to better tailor current and future treatments (Ades F., et al. Journal of Clinical Oncology. 2014, 32:2794-2803; Prat A, et al. Mol Oncol. 2011, 5:5-23, Prat A, et al. Nat Rev Clin Oncol 2012, 9:48-57).

Over the last decade, the two main molecular subtypes in HR+/HER2− disease (i.e., Luminal A and B) have been extensively investigated and identified in molecular characterization studies (The Cancer Genome Atlas (TCGA): Comprehensive molecular portraits of human breast tumours. Nature. 2012, 490:61-70; Prat A, et al. Journal of Clinical Oncology. 2013. 31:203-209; Perou C M, et al. Nature. 2000, 406:747-752). Luminal A subtype tumors have a better prognosis at 5- and 10-year follow-up compared to Luminal B subtype tumors regardless of the conventional clinical-pathological variables (e.g., tumor size and nodal status) and (neo)adjuvant treatment (i.e., endocrine therapy and chemotherapy) ((The Cancer Genome Atlas (TCGA): Comprehensive molecular portraits of human breast tumours. Nature 2012; 490:61-70; Prat A, et al. Journal of Clinical Oncology. 2013, 31:203-209, Martin M, et al. Breast Cancer Research and Treatment. 2013, 138:457-466, Prat A, et al. Journal of the National Cancer Institute. 2014, 106). In terms of sensitivity to treatment, Luminal A subtype tumors achieve pathological complete response (pCR) rates that are significantly lower than those of Luminal B subtype tumors after neoadjuvant chemotherapy with several drugs (Usary J, et al. Clinical Cancer Research. 2013, 19:4889-4899; von Minckwitz G, et al. Journal of Clinical Oncology. 2012, 30:1796-1804; Prat A, et al. Breast Cancer Research and Treatment. 2012, 135:301-306; Prat A, et al. BMC Medicine. 2015, 13:1-11). However, the difference in sensitivity to endocrine therapy between the two luminal subtypes is less clear-cut (Ellis M J, et al. Journal of Clinical Oncology. 2011, 29:2342-2349; Dunbier A K, et al. Steroids. 2011, 76:736-740).

A 5- to 10-year adjuvant endocrine therapy is recommended today for all patients with HR+/HER2− early breast cancer, whereas chemotherapy is recommended for patients with intermediate and high risk tumors (Goldhirsch A, et al. Personalizing the treatment of women with early breast cancer: highlights of the St Gallen International Expert Consensus on the Primary Therapy of Early Breast Cancer 2013. Annals of Oncology). However, the relationship between therapy and risk warrants continuous research taking into account that risk is associated with both factors related to tumor biology and clinical-pathological characteristics such as tumor size and nodal status, whereas treatment responsiveness is usually considered independent of clinical-pathological factors.

As a result, there is still a need for tools for identifying heterogeneous subgroups with HR+/HER2− breast cancer having a different prognosis and different sensitivity to treatment for the purpose of selecting the type and the degree of treatment most suitable for the patient.

### DESCRIPTION OF THE INVENTION

Hormone receptor-positive (HR+) breast cancer is clinically and biologically heterogeneous and there is a need to identify subgroups with a different prognosis and sensitivity to treatment. The present invention describes the development and clinical validation in multiple studies of a gene expression-based predictor which uses the known analysis PAM50 that is associated with response to chemotherapy and endocrine therapy in early breast cancer beyond the risk of relapse (ROR) and intrinsic PAM50 subtypes. The clinical utility of this chemoendocrine score (CES) predictor based on PAM50 lies in the group with an intermediate PAM50 ROR, where the proportion of each CES group (sensitive to endocrine therapy, intermediate, and sensitive to chemotherapy) is greater than 25%.

CES is a unique gene signature capable of measuring sensitivity to chemotherapy and endocrine therapy in HR+/HER2− breast cancer beyond the intrinsic subtype, other gene signatures, and standard pathological variables. CES may have considerable clinical value in patients with intermediate risk HR+/HER2− disease in which the benefit of adjuvant multi-agent chemotherapy cannot be determined. When the CES value is equal to or greater than 0.7, it indicates that said patient responds to endocrine treatment (CES-E), and when CES is equal to or less than 0.3, it indicates that said patient responds to chemotherapy (CES-C). It must be pointed out that there is also a CES-U group that seems to be rather ill-defined and decisions relating to the need for chemotherapy may be difficult to make.

The results of the present invention are the first in confirming, in randomized cases, an inverse relationship between sensitivity to endocrine therapy and chemotherapy in estrogen receptor-positive (ER+) breast cancer. Earlier evidence has shown an inverse relationship between proliferation and ER (estrogen receptor)-related biological processes in terms of sensitivity to endocrine therapy and chemotherapy in ER+ breast cancer. For example, two independent studies demonstrated an inverse correlation between a score of 200 ER-related genes, or between the expression of TAU, an ER-related gene, and sensitivity to endocrine therapy and chemotherapy (Symmans W F, et al. Journal of Clinical Oncology. 2010, 28:4111-4119; Andre F, et al. Clinical Cancer Research. 2007, 13:2061-2067). Likewise, the high recurrence score measured by Oncotype DX (Genomic Health, Inc., Redwood, Calif.) predicted little or no benefit from adjuvant tamoxifen treatment in the NSABP-B14 trial, but it also predicted at the same a considerable benefit from CMF (cyclophosphamide, methotrexate, fluorouracil) adjuvant chemotherapy in the NSABP-B20 trial (Paik S, et al. New England Journal of Medicine. 2004, 351:2817-2826; Paik S, et al. Journal of Clinical Oncology. 2006, 24:3726-3734). These results coincide with the results of the present invention which show that almost all patients suffering a disease with a high ROR are identified as CES-C. However, this new method also highlights that, in the disease with a high ROR/CES-C, not all the ER+/HER2− samples are luminal (i.e., Luminal A or B) given that non-luminal disease (i.e., Basal-like and HER2-enriched) can also be identified. According to the results of the present invention, the benefit of chemotherapy in tumors with a high ROR/non-luminal tumors in HR+/HER2− disease is probably even greater than in tumors with a high ROR/luminal B tumors.

The results of this new method also indicate that the intrinsic biology of Basal-like versus Luminal A constitutes the main driving force in sensitivity to endocrine therapy and chemotherapy in ER+/HER2− disease. To reflect both biological states in each sample, the correlation coefficients of each of the samples with respect to both PAM50 centroids (i.e., Luminal A and Basal-like) have been calculated and both coefficients are then subtracted (the correlation coefficient of luminal A (CC Luminal A) versus the correlation coefficient of Basal-like (CC Basal-Like)). Accordingly, instead of choosing a genetic profile (e.g., a proliferation-based profile) from the many profiles which can discriminate between both subtypes in one way or another, it was decided to incorporate into a score, for each tumor, the Basal-like versus luminal A intrinsic state of each tumor identified by the PAM50 subtype predictor. It must be pointed out that PAM50 genes were originally selected for their capacity to reflect the intrinsic biology shown by 1900 genes (the so-called intrinsic gene list). In fact, in the TCGA, the intrinsic subtype defined by PAM50 reflected the vast majority of the biological diversity shown by the analyzed molecular dataset (TCGA: Comprehensive molecular portraits of human breast tumours. Nature. 2012, 490:61-70).

From a clinical perspective, the data of this new method corroborates the current guidelines for the systemic treatment of HR+/HER2− early breast cancer. On one hand, patients with a low ROR score and a low cancer burden (i.e., <10% of risk of distant relapse at 10 years) are recommended to only receive endocrine therapy (Harris L N, et al. Journal of Clinical Oncology. 2016). In fact, the results shown with this new method indicate that these patients have tumors with high sensitivity to endocrine therapy and low sensitivity to chemotherapy. On the other hand, patients with high risk HR+/HER2 disease are recommended to receive treatment with endocrine therapy and chemotherapy. According to the data of this new method, this group is the only group with high chemotherapy benefit and low endocrine therapy benefit. With respect to endocrine therapy in this group, the main problem lies in the unavailability of survival data indicating that CES-C tumors do not benefit at all from endocrine therapy. Therefore, the removal of a potentially effective treatment strategy such as endocrine therapy in a patient with an ER+ tumor (as defined by the ASCO/CAP [American Society of Clinical Oncology/College of American Pathologist] guidelines) which is identified as CES-C or high ROR is not recommended today, although in patients whose tumors contain low ER levels (1% to 10%), the ASCO/CAP recommends discussing the advantages and drawbacks of endocrine therapy. Organizing a large randomized adjuvant trial with the participation of thousands of patients to answer this particular question is rather unlikely.

Although CES has minimum clinical implications in low and high risk HR+/HER2− disease, the observation that intermediate risk HR+/HER2− disease, which represents ˜30% of newly diagnosed breast cancers, is biologically heterogeneous and exhibits a wide range of chemotherapy sensitivities may have implications in the interpretation of two ongoing prospective clinical trials. In the phase III TailorX trial, 4500 patients with node-negative HR+/HER2− early breast cancer and an intermediate recurrence score were randomized to receive adjuvant chemotherapy or not receive chemotherapy. According to the analysis conducted by the present inventors, this intermediate group may be made up of at least 3 groups with different chemotherapy sensitivities. It must be pointed out that the CES-U group seems to be rather ill-defined and decisions relating to the need for chemotherapy may be difficult to make. A similar situation may occur in the phase III RxPONDER clinical trial in which patients with low or intermediate risk HR+/HER2− early breast cancer and between 1 and 3 positive lymph nodes are being randomized to receive adjuvant chemotherapy or not. A possible explanation is that the recurrence score of Oncotype DX, as well as other gene expression-based prognostic tests, such as PAM50 ROR or MammaPrint43, have been specifically designed or obtained for predicting results and not intrinsic tumor biology or sensitivity to treatment. Although a high negative correlation is observed between ROR (risk) and CES (sensitivity to medicinal products), there are considerable differences between them at the individual level (˜40% discordance).

There are several exceptions in the study of the present invention. First, it is a retrospective study in heterogeneous patient populations and the results must be confirmed in one or more prospective clinical trials. Second, although the data herein presented validates CES from a clinical perspective, it will be necessary to perform a subsequent analytical validation since the research-based PAM50 version was used in most of the datasets, except for the Malaga set. However, the fact that CES (as a continuous variable with the 2 cut-off points) predicted pCR in the Malaga set indicates that analytical validation of this biomarker is feasible. Third, it is impossible to evaluate the association of CES with the survival data from a randomized clinical trial of adjuvant chemotherapy versus the absence of adjuvant chemotherapy or adjuvant endocrine therapy versus the absence of adjuvant endocrine therapy. Therefore, the predictive value of these profiles was only evaluated in neoadjuvant therapy in which the different tumor response variables were evaluated, most of which have been related with patient survival (Ogston K N, et al. The Breast. 2003, 12:320-327; Cortazar P, et al. The Lancet. 2014, 384:164-172). Fourth, some of the profiles evaluated in the MDACC-based dataset, such as Oncotype DX recurrence score or genomic grade index, originated from microarray-based data, and therefore are not commercially available versions. Fifth, it was not possible to demonstrate a constant association of CES with endocrine response in HR+ disease after excluding HER2-positive cases. In the Edinburgh dataset, HER2 status was not available for all the patients. Although an ERBB2 expression-based subrogate definition of HER2 status is derived, demonstrating that CES is independently associated with response, this was not previously specified and did not meet REMARK (“REporting recommendations for tumour MARKer prognostic studies”, McShane L M et al. Br J Cancer. 2005 Aug. 22; 93(4): 387-391) guidelines. Furthermore, the association of CES with endocrine response failed to achieve statistical significance (p=0.09) in patients with HR+/HER2− disease in the Marsden dataset. Finally, patients from each of the datasets received different anthracycline/taxane-based chemotherapy regimens, programs, and doses, and therefore the capacity of the profiles for predicting response to specific chemotherapy drugs or treatment regimens could not be evaluated.

Another important consideration of this new method is that the purpose is not to identify one or more optimum cut-off points for CES, but rather to concentrate on the association of the continuous expression of CES with each variable. The main reason is that different gene expression-based platforms and protocols were used in each cohort, and therefore the normalization of a biomarker cut-off point would have been difficult to achieve and probably rather unreliable. In any case, the fact that the four contrast groups exhibit very similar associations which were found regardless of the platform/protocol used, advocates a firm conclusion.

In conclusion, CES is a unique gene signature capable of measuring sensitivity to chemotherapy and endocrine therapy in HR+/HER2− breast cancer beyond the intrinsic subtype, other gene signatures, and standard pathological variables. CES may have considerable clinical value in patients with intermediate risk HR+/HER2− disease in which the benefit of adjuvant multi-agent chemotherapy cannot be determined.

According to the information provided in relation to this new method, a first aspect relates to an in vitro method for predicting whether a patient with breast cancer will respond to chemotherapy or to endocrine therapy in an HR+/HER2− sample isolated from the patient classified in the group with an intermediate ROR by means of the PAM50 kit, which method consists of:
a) obtaining with the PAM50 kit the correlation coefficient corresponding to the sample classified as an intrinsic Luminal A subtype and the correlation coefficient corresponding to the sample classified as an intrinsic Basal-like subtype, in the isolated sample, and
b) obtaining the chemoendocrine score (CES) by subtracting the correlation coefficient corresponding to the sample classified as a Basal-like subtype from the correlation coefficient corresponding to the sample classified as a Luminal A subtype (CES=CC Luminal A−CC Basal-Like);
   wherein CES equal to or greater than 0.7 indicates that said patient responds to endocrine treatment (CES-E), and wherein CES equal to or less than 0.3 indicates that said patient responds to chemotherapy (CES-C).

In a preferred embodiment of the first aspect of this new method, the isolated sample is a biopsy sample.

A second aspect of this new method relates to the in vitro use of CES for predicting whether a patient with breast cancer will respond to chemotherapy or endocrine therapy in an isolated HR+/HER2− sample of the patient classified in the group with an intermediate ROR by means of the PAM50 kit.

In a preferred embodiment, CES is obtained by subtracting the correlation coefficient corresponding to the sample classified as a Basal-like subtype from the correlation coefficient corresponding to the sample classified as a Luminal A subtype by means of the PAM50 kit.

In another preferred embodiment of this aspect of the new method, it must be pointed out that a CES equal to or greater than 0.7 indicates that said patient is CES-E. In another preferred embodiment, a CES equal to or less than 0.3 indicates that said patient is CES-C.

In another preferred embodiment, the isolated sample is a biopsy sample.

Unless otherwise indicated, all the technical and scientific terms used herein have the same meaning as commonly understood by one skilled in the discipline to which this new method corresponds. Methods and materials that are similar or equivalent to those described herein can be used for putting this new method into practice. In the description and claims, the word “include” and variations thereof do not intend to exclude other technical features, additions, components, or steps. Other objects, advantages, and features of the new method will become apparent to those skilled in the art after having analyzed the description or will be known by means of putting the new method into practice. The following examples and drawings are provided by way of illustration and do not seek to limit this new method.

### DESCRIPTION OF THE DRAWINGS

FIG. 1. Association of gene expression with sensitivity to chemotherapy or endocrine therapy: association between the expression of each gene (n=542) and the response according to the Miller and Payne score in each GEICAM 2006-03 trial group. Certain main genes the expression of which is highly associated with response are shown on the right.

FIG. 2. Association of gene expression with sensitivity to chemotherapy or endocrine therapy: mean expression of the 50 main genes associated with sensitivity to endocrine therapy (top panel) and sensitivity to chemotherapy (bottom panel) in the GEICAM 2006-03 trial in different intrinsic breast cancer subtypes. The RNA sequence-based gene expression data was obtained from The Cancer Genome Atlas breast cancer project data portal (https://tcgadata.nci.nih.gov/tcga/).

FIG. 3. Association of gene expression with sensitivity to chemotherapy or endocrine therapy: CES significance and scoring.

FIG. 4. Association of CES (as a continuous variable) with response to chemotherapy or endocrine therapy in the 4 validation datasets.

FIG. 5. Association of CES with Miller and Payne score after HR+/HER2− disease chemotherapy of the Malaga cohort.

FIG. 6. Association of CES with sensitivity to endocrine therapy in the Edinburgh dataset (n=120): changes in the tumor volume of each patient and the response classification.

FIG. 7. Association of CES with sensitivity to endocrine therapy in the Edinburgh dataset (n=120): association of CES and other response variables (defined as a decrease of at least 70% in 90 days) in the overall population.

FIG. 8. Association of CES with sensitivity to endocrine therapy in the Edinburgh dataset (n=120): Association of CES and other response variables in HER2-negative disease.

FIG. 9. Prognosis (PAM50 ROR), intrinsic subtype, and CES in 6007 primary breast cancers: a CES and ROR score scatter plot colored by subtype is shown. The two horizontal lines indicate the cut-off points of each group of CES. The two vertical lines indicate the cut-off points of each group of PAM50 ROR.

FIG. 10. Number of patients in each group of CES based on ROR. Each bar is colored according to the subtype.

FIG. 11. Survival results in HR+ early breast cancer with an intermediate ROR: node-negative disease treated without systemic adjuvant therapy.

FIG. 12. Survival results in HR+ early breast cancer with an intermediate ROR: node-negative and node-positive disease treated only with adjuvant tamoxifen.

FIG. 13. Survival results in HR+ early breast cancer with an intermediate ROR: node-positive disease treated with adjuvant chemotherapy and endocrine therapy in the GEICAM/9906 clinical trial.

FIG. 14. Survival results in HR+ early breast cancer with an intermediate ROR: node-negative and node-positive disease treated with neoadjuvant chemotherapy and adjuvant endocrine therapy.

### EXAMPLES

Methods and Materials

GEICAM/2006-03 Clinical Trial

Pre-treatment core needle biopsy samples from patients recruited in the luminal cohort of the phase II GEICAM/2006-03 (NCT00432172) clinical trial with neoadjuvant therapy were evaluated (Alba E, et al. Annals of Oncology. 2012). In this study, 95 patients with estrogen receptor (ER)-positive breast cancer (Allred 3-8), progesterone receptor (PR)-positive breast cancer (Allred 3-8), HER2− breast cancer (according to the ASCO/CAP guidelines (Wolff A C, et al. Journal of Clinical Oncology 25:118-145, 2006)), and cytokeratin 8/18-positive breast cancer, were randomized to receive neoadjuvant chemotherapy or endocrine therapy for 24 weeks. Chemotherapy consisted of 90 mg/m² of epirubicin administered intravenously (i.v.) in combination with 600 mg/m² of cyclophosphamide i.v. every 21 days for 4 cycles, followed by 100 mg/m² of docetaxel administered intravenously every 21 days for 4 cycles. Endocrine therapy consisted of 25 mg of exemestane administered orally once a day. Pre-menopausal patients received 3.6 mg of goserelin administered subcutaneously every 28 days in 6 doses. After neoadjuvant treatment, the patients were subjected to mastectomy or conservation surgery plus axillary lymph node dissection (unless previous biopsy of the sentinel lymph node showed a negative result).

Pathological Response Endpoint in the GEICAM 2006-03 Trial

The Miller and Payne histological classification system with a 5-point scale was used (Ogston K N, et al. The Breast. 2003, 12:320-327) to measure tumor response. This classification system consists of a 5-point scale which focuses on reduction of tumor cellularity in breast tumor treated (in surgery) in comparison with pre-treatment samples. Grade 1: no changes in overall cellularity. Grade 2: up to 30% reduction of cellularity. Grade 3: between 30% and 90% estimated reduction in tumor cells. Grade 4: more than 90% reduction of tumor cells. Grade 5: pathological complete response (pCR). Ductal carcinoma in situ may be present. In this study, the Miller and Payne score was reduced to a 3-point scale to obtain a fair number of cases in each category and group: absence of response (grades 1 and 2), intermediate response (grade 3), and high response (grades 4 and 5).

GEICAM 2006-03 Gene Expression Analysis

Sixty-three of 95 pre-treatment tumor samples were available for gene expression analyses. For each sample, a formalin-fixed paraffin-embedded (FFPE) breast tissue section was first examined with hematoxylin and eosin staining to confirm the diagnosis and determine the tumor area. Two tumor tissue-enriched, 1 mm cores were obtained from the original tumor block and at least ˜100 ng of total RNA were purified for measuring the expression of 543 breast cancer-related genes by means of the nCounter platform (Nanostring Technologies, Seattle, Wash., USA). A log 2 transformation of the data was performed and the data was normalized by means of 5 constitutive genes (ACTB, MRPL19, PSMC4, RPLP0, and SF3A1) and 14 negative and positive controls by means of the nCounter platform (Nanostring Technologies, Seattle, Wash., USA) (GK Geiss, et al. Nat Biotech. 2008, 26:317-325).

Independent/Contrast Datasets

The gene expression and response data from 4 independent neoadjuvant therapy datasets was evaluated (Dunbier A K, et al. Steroids. 2011, 76:736-740; Hatzis C, et al. Jama. 2011, 305:1873-1881; Prat A, et al. Clinical Cancer Research. 2015; Dunbier A K, et al. Journal of Clinical Oncology. 2010, 28:1161-1167; Smith I E, et al. Journal of Clinical Oncology. 2007, 25:3816-3822; Turnbull A K, et al. Journal of Clinical Oncology. 2015). The gene expression and survival data from 4 independent datasets of patients with early breast cancer was evaluated (Prat A, et al. Journal of Clinical Oncology. 2013, 31:203-209; Hatzis C, et al. Jama. 2011, 305:1873-1881; Fan C, et al. BMC Medical Genomics. 2011, 4:1-15; Prat A, et al. Annals of Oncology. 2012, 23:2866-2873).

Hatzis Independent Dataset

The publicly available microarray gene expression-based dataset (GSE25066) reported by Hatzis et al. (Hatzis C, et al. Jama. 2011, 305:1873-1881) which includes 508 patients (272 with HR+/HER2− disease) treated with neoadjuvant multi-agent chemotherapy in various research protocols is evaluated: LAB99-402, USO-02-103, 2003-0321, and I-SPY-1. Most of the patients (96.4%) received anthracycline/taxane-based sequential regimens. Gene expression of pre-treatment samples was analyzed and post-chemotherapy tumor response data (i.e., pCR in breast or axilla versus the absence of response) for 488 patients (260 with HR+/HER2− disease) was available.

Malaga Independent Dataset

As described previously (Prat A, et al. Clinical Cancer Research, 2015), a total of 216 locally evaluated HR+/HER2− tumor samples kept in a biobank obtained from an independent multi-center Spanish cohort of patients with breast cancer was selected. A standard neoadjuvant chemotherapy regimen consisting of 8-10 cycles of anthracyclines and taxanes was prescribed to all the patients. Pre-treatment tumor samples were evaluated by means of standard PAM50 analysis (PROSIGNA®) through the nCounter diagnostic platform. Gene expression of the pre-treatment samples was analyzed in Hospital Universitario de Malaga, Nanostring Technologies normalized the data, and CES was applied in VHIO (“Vall d'Hebron Institute of Oncology”, Barcelona) blinded from clinical data. Tumor response data (i.e., pCR in breast/axilla versus the absence of response) for 180 patients was available. Furthermore, response data according to the Miller and Payne score for 171 patients was available.

Marsden Independent Dataset

The RNA profiles (HumanWG-6 v2 Expression BeadChips [Illumina, San Diego, Calif.]) obtained from pre-treatment core needle tumor biopsies of 103 post-menopausal patients with ER+ primary breast cancer treated with neoadjuvant anastrozole for 16 weeks in a phase II clinical trial are evaluated (Dunbier A K, et al. Steroids. 2011, 76:736-740; Dunbier A K, et al. Journal of Clinical Oncology. 2010, 28:1161-1167; Smith I E, et al. Journal of Clinical Oncology, 2007, 25:3816-3822). A subgroup of patients received gefitinib for the first 2 weeks; however, the addition of gefitinib to anastrozole did not have any additional clinical or biological effect on Ki67. Clinical tumor response (complete and partial response versus stable and progressive disease) was used as a variable. CES was calculated in the IDIBAPS (Institut d'Investigacions Biomèdiques August Pi i Sunyer, Barcelona) blinded from clinical data.

Edinburgh Independent Dataset

The RNA profiles (HumanWG-6 v2 Expression BeadChips [Illumina, San Diego, Calif.]) and Affymetrix U133A 2.0 [Santa Clara, Calif., USA]) obtained from pre-treatment core needle tumor biopsies of 120 post-menopausal patients with ER+ primary breast cancer treated with neoadjuvant anastrozole for at least 12 weeks are evaluated (Turnbull A K, et al. Journal of Clinical Oncology, 2015). Response was evaluated by means of ultrasound examination. Clinical tumor response was defined as a reduction in tumor volume of at least 70% for 90 days of treatment. Raw gene expression data can be found in Gene Expression Omnibus (GSE55374 and GSE20181). HER2 clinical status was available in 45 cases. HER2+ cases exhibited greater ERBB2 expression in comparison with HER2− cases. The 80th percentile is used as the cut-off point to define HER2 positivity in those cases without HER2 clinical status. A total of 89 cases were HER2−.

Intrinsic Subtype Assignment

All the tumors were assigned to an intrinsic breast cancer molecular subtype (Luminal A, Luminal B, HER2-enriched, Basal-like) and the normal group by means of research-based PAM50 subtype predictor (Parker J S, et al. J Clin Oncol. 2009, 27:1160-1167; Nielsen T O, et al. Clin Cancer Res. 2010, 16:5222-5232), except for the Malaga cohort in which a standard, commercially available PAM50 analysis based on nCounter was used. Before subtyping, each dataset was normalized as reported previously (Prat A, et al. Br J Cancer. 2014, 111:1532-1541, 2014), except for the Malaga cohort which was normalized by means of Nanostring according to its algorithm. It must be pointed out that the microarray-based Edinburgh dataset only consists of ER+ samples and it was not possible to suitably focus on the so-called intrinsic subtyping (Prat A, et al. Nat Rev Clin Oncol. 2012, 9). In this dataset, CES was evaluated as a continuous variable as it was not affected by centering.

Adjuvant Dataset

Gene expression and survival data from 4 independent datasets of patients with early breast cancer with an intermediate ROR was evaluated (Wolff A C, et al. Journal of Clinical Oncology, 2006, 25:118-145; Hatzis C, et al. Jama. 2011, 305:1873-1881; Fan C, et al. BMC Medical Genomics. 2011, 4:1-15; Prat A, et al. Annals of Oncology. 2012, 23:2866-2873). The first dataset is the MDACC-based dataset (MD Anderson Cancer Center) described above in which a distant relapse-free survival was recorded (Hatzis C, et al. Jama. 2011, 305:1873-1881, 2011). All the patients received neoadjuvant chemotherapy and endocrine therapy. The second dataset is a previously published combined cohort of 1318 patients with HR+ disease treated only with adjuvant tamoxifen (Prat A, et al. Annals of Oncology. 2012, 23:2866-2873). The third dataset is a previously published combined cohort of patients who did not receive any systemic adjuvant therapy (Fan C, et al. BMC Medical Genomics. 2011, 4:3). Finally, samples from the GEICAM/9906 clinical trial in which all the patients received adjuvant multi-agent chemotherapy and endocrine therapy are analyzed as described previously (Prat A, et al. Br J Cancer. 2014; 111:1532-1541) (Prat A, et al. J Clin Oncol. 2013, 31:203-9).

Combined Primary Breast Cancer Cohort

To evaluate the relationship between the PAM50 subtypes, prognosis (ROR-P, ROR-Prognosis) and CES, the PAM50 data from 7 independent cohorts described previously (TCGA: Comprehensive molecular portraits of human breast tumours. Nature, 2012 490:61-70; Prat A, et al. Journal of Clinical Oncology. 2013, 31:203-209; Prat A, et al. Breast Cancer Research and Treatment. 2012, 135:301-306; Hatzis C, et al. Jama. 2011, 305:1873-1881; Curtis C, et al. Nature. 2012, 486:346-352; Horak C E, et al. Clinical Cancer Research. 2013, 19:1587-1595; Fan C, et al. BMC Medical Genomics. 2011, 4:3), representing a total of 6007 primary tumor samples, were combined. CES in each cohort was evaluated and a combined matrix was created.

Statistical Analysis

The biological analysis of the gene lists was performed with the DAVID 6.7 annotation tool (Dennis G, et al. Genome Biol. 2003, 4:R60-543) using the list of 543 genes as a reference. The association between the expression of each gene and the response according to the Miller and Payne score (3 categories) was evaluated by means of a quantitative significance analysis of microarrays (SAM) (Tusher V G, et al. Proc Natl Acad Sci USA. 2001, 98:5116-5121). In both contrast datasets, the association between each variable and pCR or clinical/radiological response was evaluated by means of univariate and multivariable logistic regression analyses. The predictive performance of CES was evaluated by means of the receiver operating characteristic (ROC) curve analysis. Survival estimates were obtained from Kaplan-Meier curves and the existence of differences was verified by means of the log rank test. Univariate and multivariable Cox models were used for determining the independent prognostic significance of each variable. The reported p-values are two-sided.

Results

GEICAM/2006-03 Dataset

Sixty-three pre- and post-menopausal patients were evaluated in this study (Table 1). Most of the patients exhibited ductal carcinomas (83%), tumors measuring 2-5 cm in size (76%), grade 3 histological tumors (59%), node-negative (54%), and a luminal subtype determined by means of PAM50 (84%). After chemotherapy, Luminal B subtype tumors showed a higher response according to the Miller and Payne score than Luminal A subtype tumors (mean of 2.0 versus 1.4, P=0.048). However, no difference was observed in the response between the two luminal subtypes after endocrine therapy (P=0.407). Furthermore, no statistically significant interaction (P=0.429) was observed between subtype and treatment (endocrine therapy versus chemotherapy) in tumor response. It must be pointed out that the only patient who achieved pCR (i.e., grade 5 according to the Miller and Payne score) had a Basal-like tumor and was included in the chemotherapy group.

TABLE 1
Clinical-pathological characteristics and distribution
of subtypes in the GEICAM 2006-03* study.

| | CT | % | ET | % | P-value |
|---|---|---|---|---|---|
| Num. | 32 | — | 31 | — | — |
| Age (mean) | 53.7 | — | 52.3 | — | 0.596 |
| Menopausal status | | | | | |
| Pre-menopausal | 14 | 44% | 14 | 45% | 1.000 |
| Post-menopausal | 18 | 56% | 17 | 55% | |
| Tumor stage | | | | | |
| T1 | 1 | 3% | 2 | 6% | |
| T2 | 23 | 72% | 25 | 81% | 0.420 |
| T3 | 8 | 25% | 4 | 13% | |
| Mean tumor size (cm) | 4.2 | | 3.8 | | 0.278 |
| Nodes | | | | | |
| N0 | 15 | 47% | 19 | 61% | |
| N1 | 16 | 50% | 11 | 35% | 0.501 |
| N2 | 1 | 3% | 1 | 3% | |
| Grade | | | | | |
| G1 | 0 | 0% | 0 | 0% | |
| G2 | 8 | 25% | 6 | 19% | |
| G3 | 18 | 56% | 19 | 61% | 0.862 |
| G4 | 6 | 19% | 6 | 19% | |
| Histological type | | | | | |
| Ductal | 26 | 81% | 26 | 84% | |
| Lobular | 2 | 6% | 2 | 6% | 1.000 |
| Others | 4 | 13% | 3 | 10% | |
| Ki-67 IHQ (mean) | 31.1 | | 33.5 | | 0.720 |
| Miller-Payne score (mean) | 2.6 | | 2.2 | | 0.124 |
| PAM50 | | | | | |
| Luminal A | 16 | 50% | 13 | 42% | 0.564 |
| Luminal B | 11 | 34% | 13 | 42% | |
| HER2-E | 0 | 0% | 1 | 3% | |
| Basal-like | 2 | 6% | 0 | 0% | |
| Normal-like | 3 | 9% | 4 | 13% | |

*CT, chemotherapy arm; ET, endocrine therapy arm

Association of Gene Expression with Sensitivity to Treatment

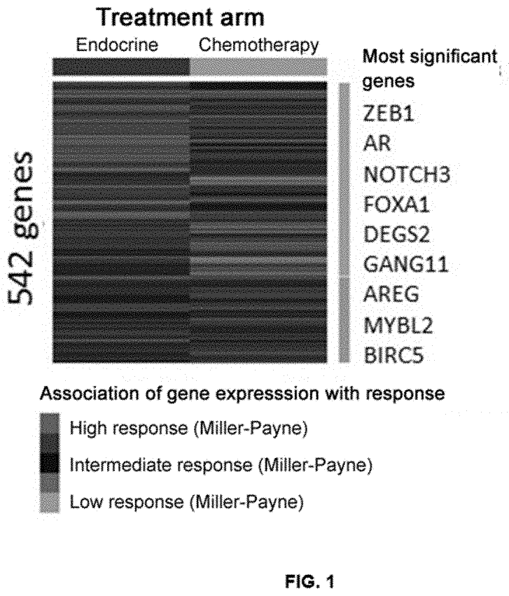

To understand the biology associated with sensitivity to chemotherapy or endocrine therapy in HR+/HER2− disease, the association between the expression of 543 breast cancer-related genes and the response according to the Miller and Payne score in each treatment group is investigated. The high expression of 70 (12.9%) and 17 (3.1%) genes was significantly (P<0.05 not corrected for multiple comparisons) related to the response after endocrine therapy and chemotherapy, respectively. The list of genes associated with the response to endocrine therapy was enriched in the following biological processes: vascularization (e.g., AKT1 and catenin beta 1), conduit development (e.g., FOXA1 and gremlin 1), and cell growth (e.g., androgen receptor and fibroblast growth factor receptor 1). Moreover, the list of genes associated with the response to chemotherapy was enriched in the cell cycle (e.g., EXO1 and MKI67) and the extracellular matrix (e.g., netrin 4 and thrombospondin 1). Interaction between the expressions of each gene with the response to therapy (endocrine therapy versus chemotherapy) was then evaluated. It must be pointed out that 41 of the 70 genes associated with the response to endocrine therapy and 8 of the 17 genes associated with the response to chemotherapy showed a significant interaction with treatment (P<0.05 not corrected in multiple comparisons). Accordingly, the biological factors associated with endocrine sensitivity seemed to also be associated with chemotherapy resistance, and vice versa. In fact, an overall inverse pattern between the expression of most of the genes and the response to treatment was observed (FIG. 1).

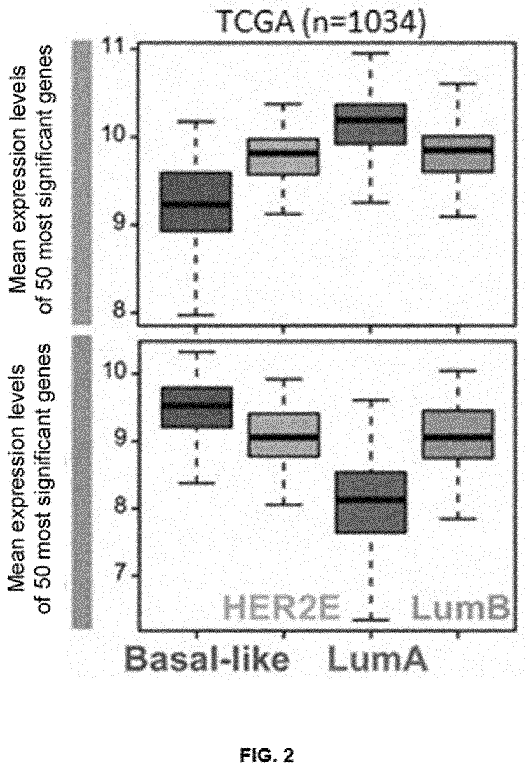

To better understand the biological factors associated with the response to treatment, the mean expression of the genes associated with high sensitivity to endocrine therapy but low sensitivity to chemotherapy or low sensitivity to endocrine therapy but high sensitivity to chemotherapy are evaluated in 1034 primary tumors exhibiting all the intrinsic breast cancer molecular subtypes (FIG. 2). The results showed that the biology associated with sensitivity to chemotherapy and endocrine therapy is primarily based on Luminal A biology (i.e., high sensitivity to endocrine therapy but low sensitivity to chemotherapy) versus Basal-like biology (i.e., low sensitivity to endocrine therapy but high sensitivity to chemotherapy).

Development of a CES Based on PAM50

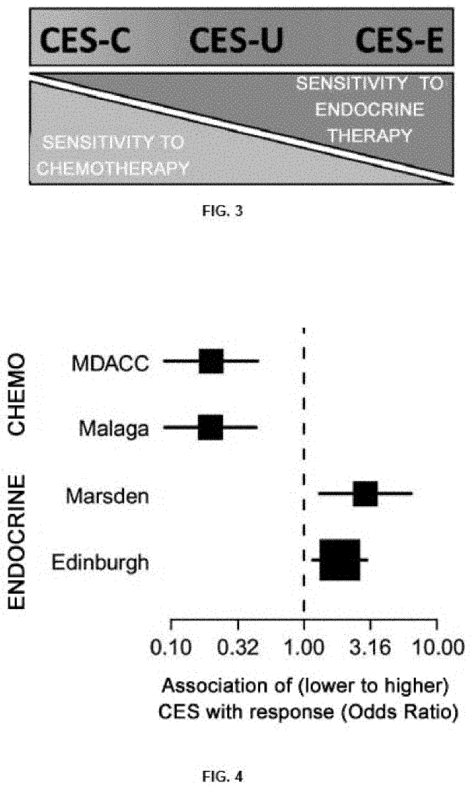

Reflecting the relative differences between Luminal A biology and Basal-like biology in HR+/HER2− may help to better predict endocrine therapy and sensitivity to chemotherapy. To reflect this biological state in each tumor, the correlation coefficients (CCs) of each sample with respect to the PAM50 Luminal A and Basal-like subtype centroids were obtained based on the PAM50 classification algorithm, and the two values were then subtracted to obtain the chemoendocrine score (CES=CC Luminal A−CC Basal-like). Samples with a positive score thereby showed greater to sensitivity to endocrine therapy than to chemotherapy, whereas samples with a negative score showed greater sensitivity to chemotherapy (CES-C, “CES-Chemo”) than to endocrine therapy (CES-E, “CES-Endocrine”) (FIG. 3). Based on the results with samples from the GEICAM 2006-03 trial, cut-off points were determined based on tertile groups (CES group-E versus CES-undetermined [CES-U], cut-off point=0.70; CES-U group versus CES-C, cut-off point=0.30). The interaction of the CES score (as a continuous variable) with treatment in the GEICAM 2006-03 trial provides certain proof of association (P=0.059).

MDACC-Based Dataset

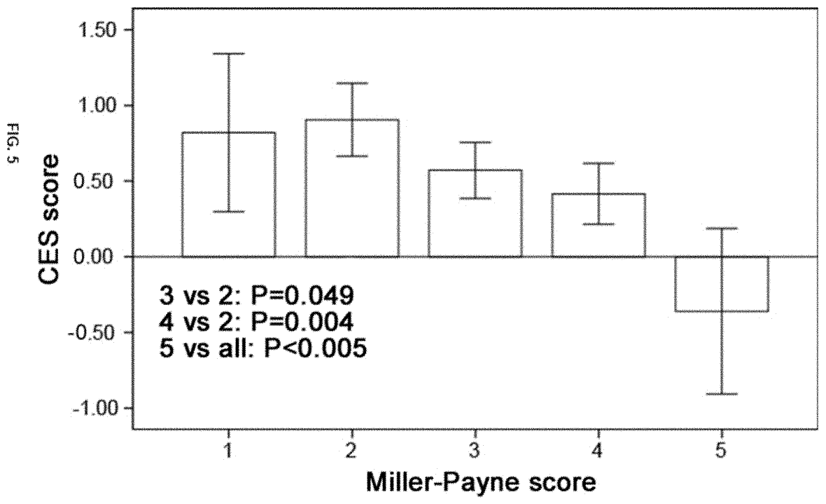

A combined dataset of 272 patients with HR+/HER2− disease treated with anthracycline/taxane-based neoadjuvant chemotherapy is evaluated in several trials with neoadjuvant therapies (FIG. 4, Table 2).

TABLE 2
Clinical-pathological characteristics and distribution
of subtypes in the 4 contrast groups*.

| | MDACC | | Malaga | | Marsden | | Edinburgh | |
|---|---|---|---|---|---|---|---|---|
| | N | % | N | % | N | % | N | % |
| Treatment | CT | | CT | | ET | | ET | |
| N | 272 | | 180 | | 103 | | 120 | |
| HER2 status^{¥} | | | | | | | | |
| HER2-negative | 272 | 100% | 180 | 100% | 89 | 86% | 31 | 69% |
| HER2-positive | 0 | 0% | 0 | 0% | 14 | 14% | 14 | 31% |
| Age (mean) | 50.1 | | 50.0 | | 53.7 | | 76.1 | |
| Menopausal status | | | | | | | | |
| Pre-menopausal | ND | | 108 | 60% | 0 | 0% | 0 | 0% |
| Post-menopausal | ND | | 72 | 40% | 103 | 100% | 120 | 100% |
| Tumor stage | | | | | | | | |
| T0-T1 | 19 | 7% | 18 | 10% | 60 | 58% | 10 | 9% |
| T2 | 142 | 52% | 115 | 67% | | | 42 | 36% |
| T3-T4 | 111 | 41% | 39 | 23% | 43 | 42% | 63 | 55% |
| Nodes | | | | | | | | |
| N0 | 96 | 35% | 67 | 37% | 61 | 59% | 86 | 72% |
| N1 | 133 | 49% | 61 | 34% | 39 | 38% | 34 | 28% |
| N2-N3 | 43 | 16% | 52 | 29% | 3 | 3% | | |
| Grade | | | | | | | | |
| G1 | 28 | 11% | 27 | 16% | 15 | 15% | 13 | 11% |
| G2 | 136 | 53% | 96 | 57% | 63 | 62% | 82 | 68% |
| G3 | 91 | 36% | 46 | 27% | 24 | 23% | 25 | 21% |
| ET response score^{§} | ND | | ND | | 53% | | 72% | |
| CT response score | | | | | | | | |
| pCR breast/axilla | 8.8% | | 6.7% | | ND | | ND | |
| PAM50 | | | | | | | | |
| Luminal A | 141 | 52% | 54 | 30% | 37 | 36% | — | — |
| Luminal B | 102 | 38% | 105 | 58% | 20 | 19% | — | — |
| HER2-E | 6 | 2% | 7 | 4% | 12 | 12% | — | — |
| Basal-like | 7 | 2% | 14 | 8% | 4 | 4% | — | — |
| Normal-like | 16 | 6% | — | — | 30 | 29% | — | — |

*ET, Endocrine therapy; CT, Chemotherapy.
^{¥}There are 75 patients in the Edinburgh dataset with undetermined HER2 clinical status.
^{§}The definition of response to ET is different between the Marsden and Edinburgh sets. Clinical tumor response (complete and partial response versus a stable and progressive disease) has been used as an endpoint in the Marsden set. Response was evaluated by means of ultrasound examination in the Edinburgh set. Clinical tumor response was defined as a decrease in tumor volume of at least 70% after 90 days of treatment.

In this dataset, 51.5%, 25.8%, and 22.7% of the samples were identified as CES-E, CES-U, and CES-C, respectively. pCR rates in CES-E, CES-U, and CES-C groups were 2.4%, 9.0%, and 23.7%, respectively (P<0.0001), and it was observed that these rates were similar even though non-luminal tumors are removed (2.2%, 8.8%, and 25.0%). The neoadjuvant chemotherapy predictive capacity of CES was independent of the clinical-pathological variables and of the intrinsic subtype (Table 3-4). Similar results were obtained when residual cancer burden was used as a variable (Tables 5-6).

TABLE 3
Association of CES with sensitivity to chemotherapy in the MDACC-based dataset.

| | | | Univariate Analysis | | | | Multivariate Analysis | | | |
|---|---|---|---|---|---|---|---|---|---|---|
| | | pCR | | Lower | Upper | | | Lower | Upper | |
| Signatures | N | score | OR | 95% | 95% | p-value | OR | 95% | 95% | p-value |
| Age (cont. | — | — | 1.0 | 0.93 | 1.02 | 0.251 | 1.0 | 0.92 | 1.02 | 0.205 |
| variable) | | | | | | | | | | |
| Tumor size | | | | | | | | | | |
| T0-T2 | 153 | 8% | 1.0 | — | — | — | 1.0 | — | — | — |
| T3-T4 | 107 | 9% | 1.1 | 0.47 | 2.63 | 0.813 | 0.6 | 0.22 | 1.70 | 0.341 |
| Nodes | | | | | | | | | | |
| N0 | 96 | 7% | 1.0 | — | — | — | 1.0 | — | — | — |
| N1 | 125 | 9% | 1.2 | 0.46 | 3.29 | 0.685 | 0.9 | 0.30 | 2.78 | 0.882 |
| N2-3 | 39 | 13% | 1.9 | 0.56 | 6.29 | 0.312 | 1.0 | 0.24 | 4.51 | 0.956 |
| Grade | | | | | | | | | | |
| 1 | 26 | 4% | 1.0 | — | — | — | 1.0 | — | — | — |
| 2 | 130 | 4% | 1.0 | 0.11 | 8.93 | 1.000 | 0.7 | 0.07 | 6.88 | 0.753 |
| 3 | 89 | 17% | 5.1 | 0.64 | 40.34 | 0.125 | 1.8 | 0.18 | 18.42 | 0.608 |
| PAM50 | | | | | | | | | | |
| Luminal A | 134 | 3% | 1.0 | — | — | — | 1.0 | — | — | — |
| Luminal B | 99 | 15% | 5.8 | 1.86 | 18.08 | 0.002 | 1.2 | 0.25 | 6.28 | 0.792 |
| HER2-E | 6 | 0% | 0.0 | — | — | 0.989 | 0.0 | — | — | 0.991 |
| Basal-like | 7 | 29% | 13.0 | 1.91 | 88.50 | 0.009 | 0.4 | 0.02 | 9.97 | 0.586 |
| Normal-like | 14 | 14% | 5.4 | 0.90 | 32.69 | 0.065 | 1.7 | 0.23 | 12.75 | 0.602 |
| CES | | | | | | | | | | |
| CES-E | 134 | 2% | 1.0 | — | — | — | — | — | — | — |
| CES-U | 67 | 9% | 4.3 | 1.04 | 17.75 | 0.044 | — | — | — | — |
| CES-C | 59 | 24% | 13.6 | 3.73 | 49.46 | <0.001 | — | — | — | — |
| CES (cont. | — | — | 0.2 | 0.08 | 0.46 | <0.001 | 0.2 | 0.03 | 0.77 | 0.022 |
| variable) | | | | | | | | | | |

TABLE 4
Association of CES with sensitivity to chemotherapy
(measured as pCR) in the MDACC-based dataset.

| | | | Univariate Analysis | | | | Multivariate Analysis | | | |
|---|---|---|---|---|---|---|---|---|---|---|
| | | pCR | | Lower | Upper | | | Lower | Upper | |
| Signatures | N | score | OR | 95% | 95% | p-value | OR | 95% | 95% | p-value |
| Age (cont. | — | — | 1.0 | 0.93 | 1.02 | 0.251 | 1.0 | 0.91 | 1.01 | 0.776 |
| variable) | | | | | | | | | | |
| Tumor size | | | | | | | | | | |
| T0-T2 | 153 | 8% | 1.0 | — | — | — | 1.0 | — | — | — |
| T3-T4 | 107 | 9% | 1.1 | 0.47 | 2.63 | 0.813 | 0.7 | 0.26 | 1.99 | 0.540 |
| Nodes | | | | | | | | | | |
| N0 | 96 | 7% | 1.0 | — | — | — | 1.0 | — | — | — |
| N1 | 125 | 9% | 1.2 | 0.46 | 3.29 | 0.685 | 0.9 | 0.31 | 2.70 | 0.861 |
| N2-3 | 39 | 13% | 1.9 | 0.56 | 6.29 | 0.312 | 0.9 | 0.20 | 3.81 | 0.846 |
| Grade | | | | | | | | | | |
| 1 | 26 | 4% | 1.0 | — | — | — | 1.0 | — | — | — |
| 2 | 130 | 4% | 1.0 | 0.11 | 8.93 | 1.000 | 0.8 | 0.09 | 7.91 | 0.866 |
| 3 | 89 | 17% | 5.1 | 0.64 | 40.34 | 0.125 | 2.43 | 0.25 | 24.13 | 0.447 |
| PAM50 | | | | | | | | | | |
| Low | 75 | 5% | 1.0 | — | — | — | 1.0 | — | — | — |
| Med | 133 | 6% | 1.1 | 0.33 | 3.91 | 0.840 | 0.5 | 0.11 | 2.08 | 0.333 |
| High | 52 | 21% | 4.8 | 1.42 | 15.93 | 0.011 | 0.7 | 0.12 | 3.61 | 0.642 |
| CES | | | | | | | | | | |
| CES-E | 134 | 2% | 1.0 | — | — | — | — | — | — | — |
| CES-U | 67 | 9% | 4.3 | 1.04 | 17.75 | 0.044 | — | — | — | — |
| CES-C | 59 | 24% | 13.6 | 3.73 | 49.46 | <0.001 | — | — | — | — |
| CES (cont. | — | — | 0.2 | 0.08 | 0.40 | <0.001 | 0.2 | 0.07 | 0.68 | 0.008 |
| variable) | | | | | | | | | | |

TABLE 5
Association of CES with sensitivity to chemotherapy (measured as
residual cancer burden [RCB]) in the MDACC-based dataset. Model A

| | | | Univariate Analysis | | | | Multivariate Analysis | | | |
|---|---|---|---|---|---|---|---|---|---|---|
| | | pCR | | Lower | Upper | | | Lower | Upper | |
| Signatures | N | score | OR | 95% | 95% | p-value | OR | 95% | 95% | p-value |
| Age (cont. | — | — | 1.0 | 0.93 | 1.02 | 0.251 | 1.0 | 0.91 | 1.01 | 0.776 |
| variable) | | | | | | | | | | |
| Tumor size | | | | | | | | | | |
| T0-T2 | 153 | 8% | 1.0 | — | — | — | 1.0 | — | — | — |
| T3-T4 | 107 | 9% | 1.1 | 0.47 | 2.63 | 0.813 | 0.7 | 0.26 | 1.99 | 0.540 |
| Nodes | | | | | | | | | | |
| N0 | 96 | 7% | 1.0 | — | — | — | 1.0 | — | — | — |
| N1 | 125 | 9% | 1.2 | 0.46 | 3.29 | 0.685 | 0.9 | 0.31 | 2.70 | 0.861 |
| N2-3 | 39 | 13% | 1.9 | 0.56 | 6.29 | 0.312 | 0.9 | 0.20 | 3.81 | 0.846 |
| Grade | | | | | | | | | | |
| 1 | 26 | 4% | 1.0 | — | — | — | 1.0 | — | — | — |
| 2 | 130 | 4% | 1.0 | 0.11 | 8.93 | 1.000 | 0.8 | 0.09 | 7.91 | 0.866 |
| 3 | 89 | 17% | 5.1 | 0.64 | 40.34 | 0.125 | 2.43 | 0.25 | 24.13 | 0.447 |
| PAM50 | | | | | | | | | | |
| Low | 75 | 5% | 1.0 | — | — | — | 1.0 | — | — | — |
| Med | 133 | 6% | 1.1 | 0.33 | 3.91 | 0.840 | 0.5 | 0.11 | 2.08 | 0.333 |
| High | 52 | 21% | 4.8 | 1.42 | 15.93 | 0.011 | 0.7 | 0.12 | 3.61 | 0.642 |
| CES | | | | | | | | | | |
| CES-E | 134 | 2% | 1.0 | — | — | — | — | — | — | — |
| CES-U | 67 | 9% | 4.3 | 1.04 | 17.75 | 0.044 | — | — | — | — |
| CES-C | 59 | 24% | 13.6 | 3.73 | 49.46 | <0.001 | — | — | — | — |
| CES (cont. | — | — | 0.2 | 0.08 | 0.40 | <0.001 | 0.2 | 0.07 | 0.68 | 0.008 |
| variable) | | | | | | | | | | |

TABLE 6
Association of CES with sensitivity to chemotherapy (measured as
residual cancer burden [RCB]) in the MDACC-based dataset. Model B

| | | | Univariate Analysis | | | | Multivariate Analysis | | | |
|---|---|---|---|---|---|---|---|---|---|---|
| | | RTB0/1 | | Lower | Upper | | | Lower | Upper | |
| Signatures | N | score | OR | 95% | 95% | p-value | OR | 95% | 95% | p-value |
| Age (cont. | — | — | 1.0 | 0.94 | 1.01 | 0.166 | 1.0 | 0.92 | 1.0 | 0.078 |
| variable) | | | | | | | | | | |
| Tumor size | | | | | | | | | | |
| T0-T2 | 127 | 18% | 1.0 | — | — | — | 1.0 | — | — | — |
| T3-T4 | 82 | 13% | 0.7 | 0.34 | 1.58 | 0.424 | 0.6 | 0.25 | 1.60 | 0.330 |
| Nodes | | | | | | | | | | |
| N0 | 82 | 22% | 1.0 | — | — | — | 1.0 | — | — | — |
| N1 | 105 | 12% | 0.5 | 0.23 | 1.1 | 0.084 | 0.3 | 0.13 | 0.81 | 0.016 |
| N2-3 | 32 | 13% | 0.5 | 0.16 | 1.6 | 0.260 | 0.3 | 0.06 | 1.16 | 0.078 |
| Grade | | | | | | | | | | |
| 1 | 24 | 4% | 1.0 | — | — | — | 1.0 | — | — | — |
| 2 | 102 | 14% | 3.3 | 0.41 | 26.0 | 0.266 | 3.0 | 0.35 | 25.90 | 0.314 |
| 3 | 71 | 24% | 7.2 | 091 | 57.6 | 0.061 | 5.6 | 0.06 | 51.47 | 0.130 |
| PAM50 | | | | | | | | | | |
| Luminal A | 115 | 10% | 1.0 | — | — | — | 1.0 | — | — | — |
| Luminal B | 80 | 21% | 2.3 | 1.04 | 5.17 | 0.040 | 0.6 | 0.16 | 2.48 | 0.537 |
| HER2-E | 6 | 17% | 1.7 | 0.18 | 15.94 | 0.635 | 0.4 | 0.02 | 7.71 | 0.566 |
| Basal-like | 4 | 25% | 2.9 | 0.28 | 29.73 | 0.380 | 0.4 | 0.02 | 8.40 | 0.522 |
| Normal-like | 14 | 29% | 3.4 | 0.93 | 12.70 | 0.064 | 1.4 | 0.28 | 7.01 | 0.687 |
| CES | | | | | | | | | | |
| CES-E | 115 | 10% | 1.0 | — | — | — | — | — | — | — |
| CES-U | 48 | 15% | 1.5 | 0.61 | 3.91 | 0.358 | — | — | — | — |
| CES-C | 45 | 31% | 3.9 | 1.63 | 9.25 | 0.002 | — | — | — | — |
| CES (cont. | — | — | 0.34 | 0.16 | 0.71 | 0.004 | 0.2 | 0.05 | 0.88 | 0.032 |
| variable) | | | | | | | | | | |

Seven expression-based gene signatures (i.e., PAM50 proliferation score, ROR-P, genomic grade index, SET [Sensitivity to Endocrine Therapy] index, chemopredictor, DLDA-30 [Diagonal Linear Discriminant Analysis-30″], and residual cancer burden [RCB] predictor) were previously described in this dataset (Hatzis C, et al. Jama. 2011, 305:1873-1881). Furthermore, a microarray-based version of the Oncotype DX recurrence score was applied (Fan C, et al. New England Journal of Medicine. 2006, 355:560-569; Paik S, et al. New England Journal of Medicine. 2004, 351:2817-2826). In this case, the capacity of CES to predict pCR in HR+/HER2− disease is evaluated in comparison with these seven gene signatures. Interestingly, CES provided the largest aROC (area-ROC) (Tables 7-15) either as a continuous variable (aROC=0.770) or as group categories (aROC=0.765). The second most predictive profile was the RCB predictor (aROC=0.740). It must be pointed out that the RCB predictor was obtained using 165 of 272 (60.7%) HR+/HER2− samples from this dataset (i.e., the training dataset). When these training samples were not taken into consideration, CES showed better performance either as a continuous variable (aROC=0.805) or as group categories (aROC=0.786) than the RCB predictor (aROC=0.640).

TABLE 7
Univariate association of CES and several signatures with
sensitivity to chemotherapy in HR+/HER2− disease
in the MDACC-based dataset (AUC = “Area Under
Curve”, GGI = “Genomic Grade Index”, RCBPRED =
“Residual Cancer Burden Predictor”).

| | | | | Univariate Analysis | | | |
|---|---|---|---|---|---|---|---|
| | | pCR | | | Lower | Upper | |
| Signatures | N | score | AUC | OR | 95% | 95% | p-value |
| CES | | | | | | | |
| CES-E | 134 | 2% | 0.765 | 1.0 | — | — | — |
| CES-U | 67 | 9% | | 4.3 | 1.04 | 17.75 | 0.044 |
| CES-C | 59 | 24% | | 13.6 | 3.73 | 49.46 | <0.001 |
| CES | — | — | 0.770 | 0.17 | 0.08 | 0.40 | <0.001 |
| GHI | — | — | 0.648 | 1.31 | 1.06 | 1.63 | 0.013 |
| GHI PROLIF | — | — | 0.663 | 1.33 | 1.08 | 1.62 | 0.007 |
| ROR-P | | | | | | | |
| Low | 75 | 5% | 0.659 | 1.0 | — | — | — |
| Med | 133 | 6% | | 1.1 | 0.33 | 3.91 | 0.840 |
| High | 52 | 21% | | 4.8 | 1.42 | 15.93 | 0.011 |
| PAM50 | — | — | 0.700 | 0.2 | 0.08 | 0.40 | <0.001 |
| PROLIF | | | | | | | |
| CHEMOPRED | | | | | | | |
| RxInsensitive | 159 | 6% | 0.597 | 1.0 | — | — | — |
| RxSensitive | 101 | 13% | | 2.2 | 0.93 | 5.23 | 0.074 |
| GGI | | | | | | | |
| Low | 126 | 3% | 0.670 | 1.0 | — | — | — |
| High | 134 | 14% | | 5.0 | 1.66 | 15.26 | 0.004 |
| SET | | | | | | | |
| HIGH | 21 | 5% | 0.525 | 1.0 | — | — | — |
| INTERM | 35 | 9% | | 1.9 | 0.18 | 19.29 | 0.597 |
| LOW | 204 | 9% | | 2.1 | 0.26 | 16.16 | 0.494 |
| RCBPRED | | | | | | | |
| RCB II/III | 159 | 3% | 0.740 | 1.0 | — | — | — |
| RCB 0/I | 101 | 19% | | 9.0 | 2.96 | 27.27 | <0.001 |
| DLDA30 | | | | | | | |
| RD | 254 | 2% | 0.511 | 1.0 | — | — | — |
| pCR | 6 | 17% | | 2.1 | 0.24 | 18.87 | 0.504 |

TABLE 8
Association of CES and PAM50 proliferation profile
with sensitivity to chemotherapy in HR+/HER2−
disease of the MDACC-based dataset.

| | | | Bivariate analysis | | | |
|---|---|---|---|---|---|---|
| Signatures | | pCR | | Lower | Upper | |
| CES | N | score | OR | 95% | 95% | p-value |
| CES-E | 134 | 2% | 1.0 | — | — | — |
| CES-U | 67 | 9% | 4.6 | 1.02 | 20.99 | 0.047 |
| CES-C | 59 | 24% | 15.8 | 2.93 | 85.36 | 0.001 |
| PAM50 | — | — | 0.82 | 0.21 | 3.28 | 0.783 |
| PROLIF | | | | | | |

TABLE 9
Association of CES and CHEMOPRED profile with
sensitivity to chemotherapy in HR+/HER2−
disease of the MDACC-based dataset.

| | | | Bivariate analysis | | | |
|---|---|---|---|---|---|---|
| | | pCR | | Lower | Upper | |
| Signatures | N | score | OR | 95% | 95% | p-value |
| CES | | | | | | |
| CES-E | 134 | 2% | 1.0 | — | — | — |
| CES-U | 67 | 9% | 5.0 | 1.18 | 20.83 | 0.029 |
| CES-C | 59 | 24% | 15.3 | 4.12 | 56.72 | <0.001 |
| CHEMOPRED | | | | | | |
| RxInsensitive | 159 | 6% | 1.00 | — | — | — |
| RxSensitive | 101 | 13% | 2.73 | 1.09 | 6.87 | 0.032 |

TABLE 10
Association of CES and proliferation component of
the Genomic Health Index (GHI; Oncotype DX recurrence
score) with sensitivity to chemotherapy in HR+/HER2−
disease of the MDACC-based dataset.

| | | | Bivariate analysis | | | |
|---|---|---|---|---|---|---|
| Signatures | | pCR | | Lower | Upper | |
| CES | N | score | OR | 95% | 95% | p-value |
| CES-E | 134 | 2% | 1.0 | — | — | — |
| CES-U | 67 | 9% | 5.5 | 1.19 | 25.18 | 0.029 |
| CES-C | 59 | 24% | 22.7 | 3.93 | 131.45 | <0.001 |
| GHI | — | — | 0.87 | 0.65 | 1.18 | 0.387 |
| Proliferation | | | | | | |

TABLE 11
Association of CES and the Genomic Grade Index
(GGI) profile with sensitivity to chemotherapy
in HR+/HER2− disease of the MDACC-based dataset.

| | | | Bivariate analysis | | | |
|---|---|---|---|---|---|---|
| | | pCR | | Lower | Upper | |
| Signatures | N | score | OR | 95% | 95% | p-value |
| CES | | | | | | |
| CES-E | 134 | 2% | 1.0 | — | — | — |
| CES-U | 67 | 9% | 3.7 | 0.79 | 17.16 | 0.097 |
| CES-C | 59 | 24% | 10.6 | 2.15 | 52.00 | 0.004 |
| GGI | | | | | | |
| Low | 126 | 3% | 1.00 | — | — | — |
| High | 134 | 14% | 1.43 | 0.35 | 5.77 | 0.615 |

TABLE 12
Association of CES and SET Index Signature with
sensitivity to chemotherapy in HR+/HER2−
disease of the MDACC-based dataset.

| | | | Bivariate analysis | | | |
|---|---|---|---|---|---|---|
| | | pCR | | Lower | Upper | |
| Signatures | N | score | OR | 95% | 95% | p-value |
| CES | | | | | | |
| CES-E | 134 | 2% | 1.0 | — | — | — |
| CES-U | 67 | 9% | 4.7 | 1.09 | 19.85 | 0.038 |
| CES-C | 59 | 24% | 15.3 | 3.99 | 59.02 | <0.001 |
| SET | | | | | | |
| HIGH | 21 | 5% | 1.00 | — | — | — |
| INTERM | 35 | 9% | 2.37 | 0.20 | 27.94 | 0.494 |
| LOW | 204 | 9% | 1.20 | 0.14 | 10.62 | 0.868 |

TABLE 13
Association of CES and RCBPRED signature with
sensitivity to chemotherapy in HR+/HER2−
disease of the MDACC-based dataset.

| | | | Bivariate analysis | | | |
|---|---|---|---|---|---|---|
| | | pCR | | Lower | Upper | |
| Signatures | N | score | OR | 95% | 95% | p-value |
| CES | | | | | | |
| CES-E | 134 | 2% | 1.0 | — | — | — |
| CES-U | 67 | 9% | 4.3 | 1.01 | 18.35 | 0.048 |
| CES-C | 59 | 24% | 11.8 | 3.14 | 44.47 | <0.001 |
| CTRPRED | | | | | | |
| CTR II/III | 159 | 3% | 1.00 | — | — | — |
| CTR 0/I | 101 | 19% | 7.97 | 2.55 | 24.94 | <0.001 |

TABLE 14
Association of CES and the DLDA30 signature
with sensitivity to chemotherapy in HR+/HER2−
disease of the MDACC-based dataset.

| | | | Bivariate analysis | | | |
|---|---|---|---|---|---|---|
| | | pCR | | Lower | Upper | |
| Signatures | N | score | OR | 95% | 95% | p-value |
| CES | | | | | | |
| CES-E | 134 | 2% | 1.0 | — | — | — |
| CES-U | 67 | 9% | 4.3 | 1.04 | 17.83 | 0.044 |
| CES-C | 59 | 24% | 13.9 | 3.79 | 51.25 | <0.001 |
| DLDA30 | | | | | | |
| RD | 254 | 2% | 1.00 | — | — | — |
| pCR | 6 | 17% | 0.72 | 0.08 | 6.75 | 0.773 |

TABLE 15
Association of CES and the ROR-P signature with
sensitivity to chemotherapy in HR+/HER2−
disease of the MDACC-based dataset.

| | | | Bivariate analysis | | | |
|---|---|---|---|---|---|---|
| | | pCR | | Lower | Upper | |
| Signatures | N | score | OR | 95% | 95% | p-value |
| CES | | | | | | |
| CES-E | 134 | 2% | 1.0 | — | — | — |
| CES-U | 67 | 9% | 5.38 | 1.15 | 25.06 | 0.032 |
| CES-C | 59 | 24% | 17.29 | 3.22 | 92.88 | <0.001 |
| ROR-P | | | | | | |
| Low | 75 | 5% | 1.00 | — | — | — |
| Med | 133 | 6% | 0.44 | 0.10 | 1.86 | 0.264 |
| High | 52 | 21% | 0.60 | 0.12 | 3.12 | 0.546 |

Malaga Dataset

A dataset of 180 patients with HR+/HER2− disease treated with anthracycline/taxane-based neoadjuvant chemotherapy is evaluated (Table 2). In this dataset, 46.1%, 16.1%, and 37.8% of the samples were identified as CES-E, CES-U, and CES-C, respectively. The pCR and RCB 0/1 rates in the CES-E, CES-U, and CES-C groups were 2.4%/9.6%, 3.4%/17.2%, and 13.2%/30.9%, respectively (P=0.022 and 0.004).

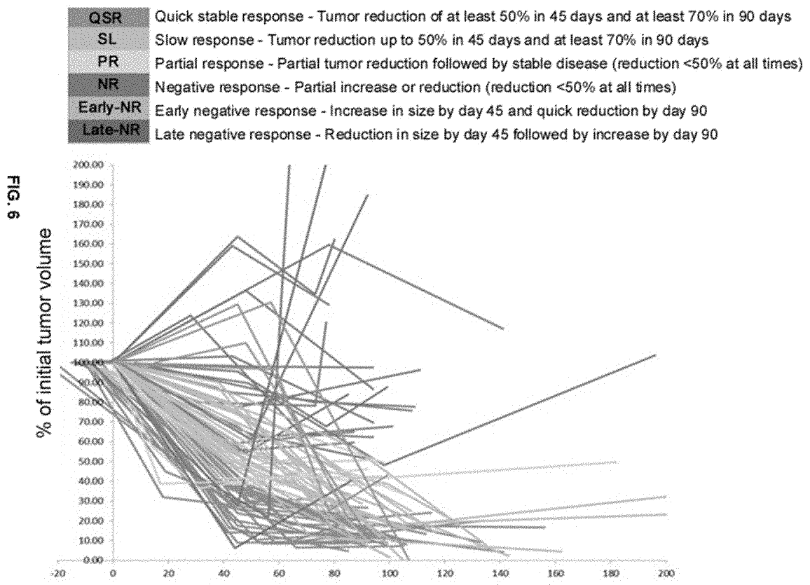

To evaluate the capacity of CES to predict the response to chemotherapy, regardless of the known clinical-pathological variables and the intrinsic subtype, a multivariate logistic regression analysis is performed using RCB (0/1 versus 2/3) as a variable because only 12 samples achieved RCB 0 (i.e., pCR) in this dataset. The results showed that CES provided independent predictive information beyond the intrinsic subtype (Table 16), Ki-67 determined by IHC (immunohistochemistry) (Table 17), and PAM50 ROR score (Table 18). The aROC of CES for predicting RCB 0/1 was 0.746. Finally, a significant association was observed between CES and the response data according to the Miller and Payne score (FIG. 5).

TABLE 16
Association of CES with sensitivity to chemotherapy in the Malaga dataset.

| | | | Univariate Analysis | | | | Multivariate Analysis | | | |
|---|---|---|---|---|---|---|---|---|---|---|
| | | pCR | | Lower | Upper | | | Lower | Upper | |
| Signatures | N | score | OR | 95% | 95% | p-value | OR | 95% | 95% | p-value |
| Age (cont. | — | — | 1.0 | 0.95 | 1.02 | 0.331 | 1.0 | 0.96 | 1.07 | 0.599 |
| variable) | | | | | | | | | | |
| Tumor size | | | | | | | | | | |
| T0-T2 | 133 | 22% | 1.0 | — | — | — | 1.0 | — | — | — |
| T3-T4 | 39 | 10% | 0.4 | 0.13 | 1.25 | 0.116 | 0.4 | 0.09 | 1.90 | 0.260 |
| Grade | | | | | | | | | | |
| 1 | 27 | 7% | 1.0 | — | — | — | 1.0 | — | — | — |
| 2 | 96 | 16% | 2.3 | 0.50 | 10.82 | 0.286 | 1.6 | 0.26 | 9.31 | 0.625 |
| 3 | 46 | 35% | 6.7 | 1.40 | 31.82 | 0.017 | 3.0 | 0.40 | 23.34 | 0.283 |
| PAM50 | | | | | | | | | | |
| Luminal A | 54 | 9% | 1.0 | — | — | — | 1.0 | — | — | — |
| Luminal B | 105 | 20% | 2.4 | 0.87 | 6.91 | 0.090 | 0.9 | 0.18 | 4.34 | 0.905 |
| HER2-E | 7 | 14% | 1.6 | 0.16 | 16.43 | 0.677 | 0.1 | 0.00 | 3.19 | 0.188 |
| Basal-like | 14 | 50% | 9.8 | 2.43 | 39.51 | 0.001 | 0.1 | 0.00 | 3.40 | 0.214 |
| CES | | | | | | | | | | |
| CES-E | 83 | 10% | 1.0 | — | — | — | — | — | — | — |
| CES-U | 29 | 17% | 2.0 | 0.58 | 6.54 | 0.277 | — | — | — | — |
| CES-C | 68 | 31% | 4.2 | 1.72 | 10.22 | 0.002 | — | — | — | — |
| CES (cont. | — | — | 0.2 | 0.09 | 0.44 | <0.001 | 0.2 | 0.07 | 0.76 | 0.016 |
| variable) | | | | | | | | | | |

TABLE 17
Association of CES and Ki-67 determined by means of
IHC with sensitivity to chemotherapy in HR+/HER2−
disease of the Malaga dataset.

| | | | Bivariate analysis | | | |
|---|---|---|---|---|---|---|
| | | RTL 0/1 | | Lower | Upper | |
| Signatures | N | score | OR | 95% | 95% | p-value |
| CES | — | — | 0.46 | 0.23 | 0.89 | 0.022 |
| Ki67 IHQ | — | — | 1.02 | 0.99 | 1.04 | 0.194 |

TABLE 18
Association of CES and PAM50 ROR with sensitivity to chemotherapy
in HR+/HER2− disease of the Malaga dataset.

| | | | Bivariate analysis | | | |
|---|---|---|---|---|---|---|
| | | RTL 0/1 | | Lower | Upper | |
| Signatures | N | score | OR | 95% | 95% | p-value |
| CES | — | — | 0.27 | 0.14 | 0.51 | <0.001 |
| PAM50 ROR | — | — | 0.98 | 0.95 | 1.02 | 0.308 |

Marsden-Based Dataset: CES and Sensitivity to Endocrine Therapy

A dataset of 103 post-menopausal patients with HR+ disease treated with anastrozole for 16 weeks with neoadjuvant therapy is evaluated (Table 2). In this dataset, 23.5%, 34.3%, and 42.2% of the samples were identified as CES-E, CES-U, and CES-C, respectively. The clinical tumor response (complete and partial response versus stable and progressive disease) was used as a variable. No pCR was observed in this dataset. The clinical tumor response rates in CES-E, CES-U, and CES-C groups were 75.0%, 48.6%, and 44.2%, respectively (P=0.043). CES was the only variable with a significant association with the response (Table 19), regardless of the HER2 status (Tables 19-20)

TABLE 19
Association of CES with sensitivity to endocrine therapy in the Marsden dataset (n = 103).

| | | | Univariate Analysis | | | | Multivariate Analysis | | | |
|---|---|---|---|---|---|---|---|---|---|---|
| | | Response | | Lower | Upper | | | Lower | Upper | |
| Signatures | N | score | OR | 95% | 95% | p-value | OR | 95% | 95% | p-value |
| Age (cont. | 103 | — | 1.0 | 0.95 | 1.04 | 0.865 | | | | |
| variable) | | | | | | | | | | |
| Tumor size | | | | | | | | | | |
| T0-T2 | 60 | 52% | 1.0 | — | — | — | | | | |
| T3-T4 | 43 | 55% | 1.0 | 0.47 | 2.29 | 0.925 | | | | |
| ARM | | | | | | | | | | |
| Arm B | 46 | 48% | 1.0 | — | — | — | | | | |
| Arm C | 57 | 56% | 1.3 | 0.53 | 2.95 | 0.467 | | | | |
| Grade | | | | | | | | | | |
| 1 | 15 | 53% | 1.0 | — | — | — | | | | |
| 2 | 63 | 52% | 1.0 | 0.32 | 3.20 | 0.965 | | | | |
| 3 | 24 | 46% | 0.8 | 0.21 | 2.97 | 0.740 | | | | |
| HER2 status | | | | | | | | | | |
| Negative | 89 | 56% | 1.0 | — | — | — | 1.0 | — | — | — |
| Positive | 25 | 28% | 0.3 | 0.07 | 0.98 | 0.058 | 0.4 | 0.10 | 1.40 | 0.170 |
| PAM50 | | | | | | | | | | |
| Basal-like | 4 | 50% | 1.0 | — | — | — | | | | |
| Luminal A | 37 | 65% | 2.0 | 0.22 | 18.41 | 0.510 | | | | |
| Luminal B | 20 | 40% | 0.67 | 0.07 | 6.50 | 0.710 | | | | |
| HER2-E | 12 | 25% | 0.38 | 0.03 | 3.78 | 0.361 | | | | |
| Normal-like | 30 | 57% | 1.31 | 0.14 | 12.12 | 0.801 | | | | |
| CES (cont. | 103 | — | 2.9 | 1.34 | 6.55 | 0.009 | 2.4 | 1.08 | 5.50 | 0.036 |
| variable) | | | | | | | | | | |

TABLE 20
Association of CES with sensitivity to endocrine therapy in the
Marsden dataset in HER2− disease (n = 89).

| | | | Univariate analysis | | | |
|---|---|---|---|---|---|---|
| | | Response | | Lower | Upper | |
| Signatures | N | score | OR | 95% | 95% | p-value |
| Age (cont. | 89 | — | 1.0 | 0.18 | 293 | 0.38 |
| variable) | | | | | | |
| Tumor size | | | | | | |
| T0-T2 | 54 | 60% | 1.0 | — | — | — |
| T3-T4 | 35 | 54% | 0.8 | 0.35 | 2.00 | 0.697 |
| ARM | | | | | | |
| Arm B | 38 | 50% | 1.0 | — | — | — |
| Arm C | 51 | 60% | 1.5 | 0.62 | 3.48 | 0.379 |
| Grade | | | | | | |
| 1 | 14 | 57% | 1.0 | — | — | — |
| 2 | 54 | 59% | 1.1 | 0.32 | 3.57 | 0.886 |
| 3 | 20 | 45% | 0.7 | 0.16 | 2.69 | 0.579 |
| PAM50 | | | | | | |
| Basal-like | 4 | 50% | 1.0 | — | — | — |
| Luminal A | 35 | 66% | 2.09 | 0.23 | 19.39 | 0.489 |
| Luminal B | 17 | 41% | 0.70 | 0.07 | 7.00 | 0.750 |
| HER2-E | 7 | 29% | 0.40 | 0.03 | 5.32 | 0.480 |
| Normal-like | 26 | 61% | 1.60 | 0.17 | 15.16 | 0.660 |
| CES (cont. | 89 | — | 2.00 | 0.90 | 4.89 | 0.090 |
| variable) | | | | | | |

Edinburgh-Based Dataset: CES and Sensitivity to Endocrine Therapy

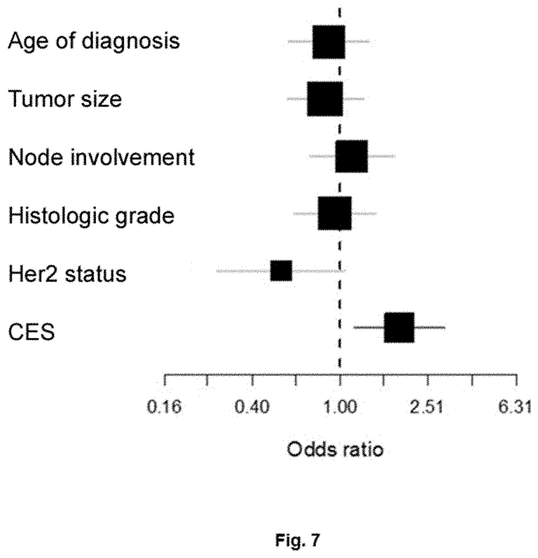

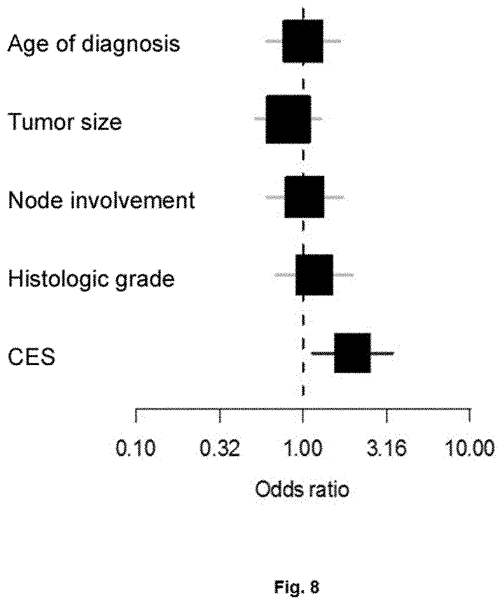

A dataset of 120 post-menopausal patients with HR+ disease treated with letrozole for at least 12 weeks with neoadjuvant therapy is evaluated (FIG. 6). Two patients out of 120 achieved complete response. Similar to the preceding results, CES as a continuous variable was the only variable with a significant association with a ≥70% reduction in tumor volume before 90 days (FIG. 7), even in HER2− disease (FIG. 8).

Prognosis, Intrinsic Subtype, and Sensitivity to Chemotherapy and Endocrine Therapy

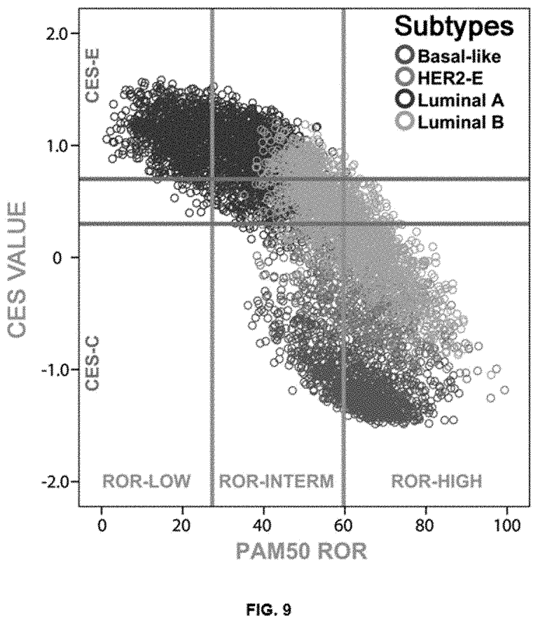

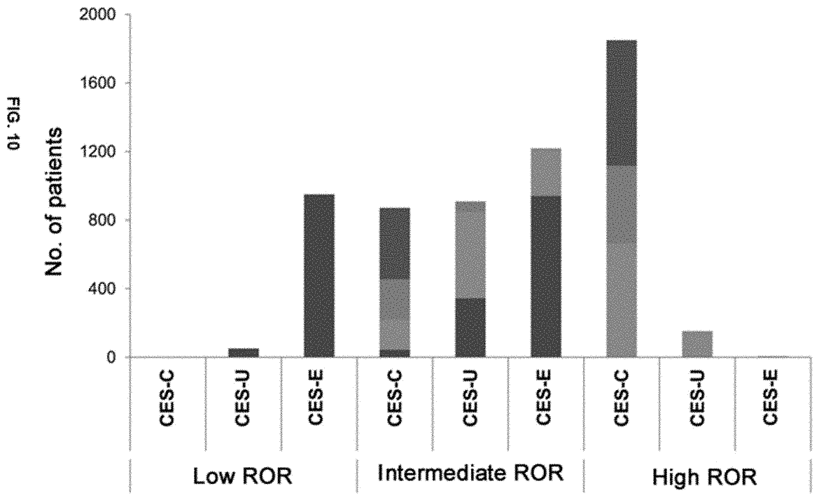

To better understand the relationship between prognosis, intrinsic biology, and sensitivity to chemotherapy and endocrine therapy, the PAM50 data of several different datasets is grouped to obtain a total of 6007 primary breast cancers representing all subtypes (FIGS. 9-10). The results showed that in the group with a low ROR, 94.9% of the cases were identified as CES-E and 100% were Luminal A subtype. In the group with a high ROR, 92.1% of the samples were identified as CES-C; non-luminal and Luminal B subtypes represented 64.3% and 35.7% of high ROR/CES-C cases, respectively.

High heterogeneity was observed in the group with an intermediate ROR. In terms of intrinsic biology, Luminal A, Luminal B, and non-luminal subtypes represented 44.4%, 31.5%, and 24.1%, respectively. In terms of sensitivity to chemotherapy and endocrine therapy, CES-E, CES-U, and CES-C represented 40.6%, 30.3%, and 29.1%, respectively. As expected, the vast majority of intermediate ROR/CES-E samples (77.3%) were Luminal A subtype.

CES Survival Results in HR+ Disease/Disease with an Intermediate ROR

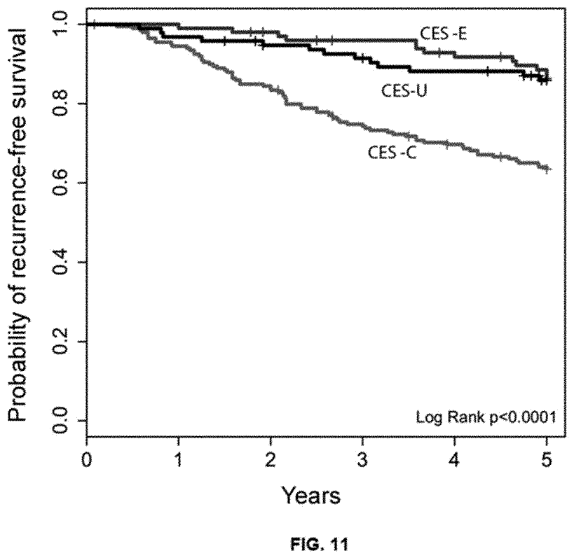

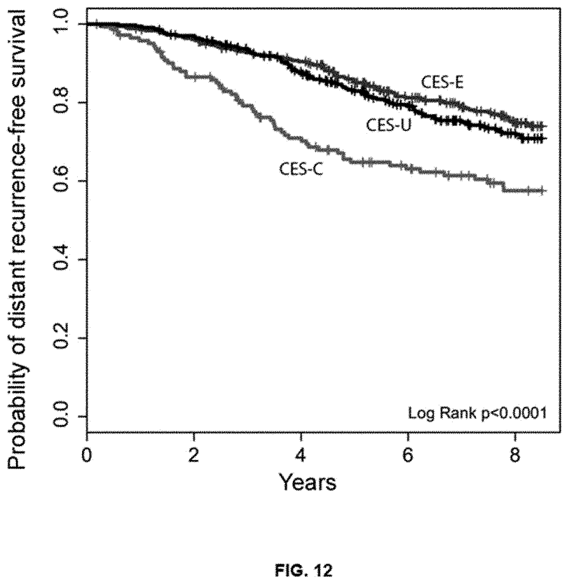

To continue studying CES value in HR+ disease/disease with an intermediate ROR, the association of CES with survival results in early HR+ breast cancer/early breast cancer with an intermediate ROR in 4 independent datasets of patients not treated with any systemic adjuvant therapy (n=189), treated only with adjuvant tamoxifen (n=846), or treated with chemotherapy and adjuvant endocrine therapy (n=322 and n=148) is evaluated. In patients with node-negative disease treated without systemic adjuvant therapy, CES (as a continuous variable or as group categories) was found to be significantly associated with distant relapse-free survival (FIG. 11). The hazard ratio between CES-C and CES-E groups was 2.68 (95% confidence interval of 0.163-0.858). Similar results were obtained in the dataset in which the patients were treated only with adjuvant tamoxifen (FIG. 12).

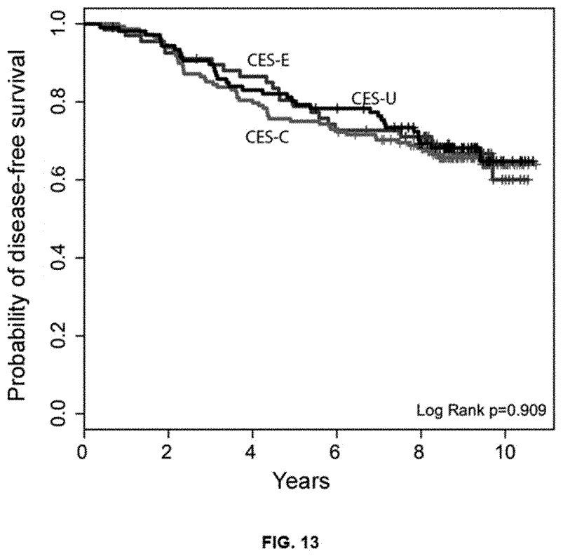

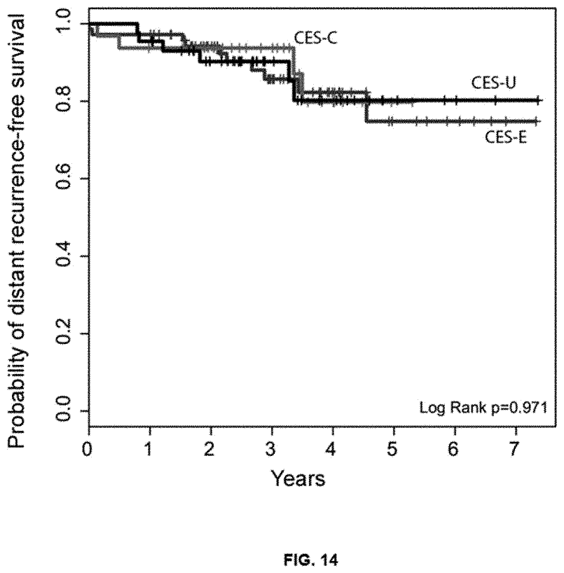

However, CES (as a continuous variable or as group categories) was not found to be significantly associated with the survival results in 2 independent cohorts of patients treated with (neo)adjuvant chemotherapy and endocrine therapy (FIGS. 13-14).

1. An in vitro method for predicting whether a patient with breast cancer will respond to chemotherapy or to endocrine therapy in an HR+/HER2− sample isolated from the patient classified in the group with an intermediate ROR by means of the PAM50 kit, which method comprises:
(a) obtaining the PAM50 kit, the correlation coefficient corresponding to the sample classified as an intrinsic Luminal A subtype and the correlation coefficient corresponding to the sample classified as an intrinsic Basal-like subtype, in the isolated sample, and
(b) obtaining the chemoendocrine score (CES) by subtracting the correlation coefficient corresponding to the sample classified as a Basal-like subtype from the correlation coefficient corresponding to the sample classified as a Luminal A subtype;
wherein CES equal to or greater than 0.7 indicates that said patient responds to endocrine treatment (CES-E), and wherein CES equal to or less than 0.3 indicates that said patient responds to chemotherapy (CES-C).

2. The in vitro method according to claim 1, wherein the isolated sample is a biopsy sample.

3. In vitro use of CES for predicting whether a patient with breast cancer will respond to chemotherapy or to endocrine treatment in an HR+/HER2− sample isolated from the patient classified in the group with an intermediate ROR by means of the PAM50 kit.

4. In vitro use according to claim 3, wherein CES is obtained by subtracting the correlation coefficient corresponding to the sample classified as a Basal-like subtype from the correlation coefficient corresponding to the sample classified as a Luminal A subtype by means of the PAM50 kit.

5. In vitro use according to claim 3, wherein a CES equal to or greater than 0.7 indicates that said patient is CES-E.

6. In vitro use according to claim 3, wherein a CES equal to or less than 0.3 indicates that said patient is CES-C.

7. In vitro use according to claim 3, wherein the isolated sample is a biopsy sample.

8. In vitro use according to claim 4, wherein a CES equal to or greater than 0.7 indicates that said patient is CES-E.

9. In vitro use according to claim 4, wherein a CES equal to or less than 0.3 indicates that said patient is CES-C.

10. In vitro use according to claim 4, wherein the isolated sample is a biopsy sample.

11. In vitro use according to claim 5, wherein the isolated sample is a biopsy sample.

12. In vitro use according to claim 6, wherein the isolated sample is a biopsy sample.
